(19) United States
(12) Patent Application Publication
(10) Pub. No.: US 2007/0027475 A1
(43) Pub. Date: Feb. 1, 2007

(54) Scaled suture thread

(75) Inventor: Anthony C. Pagedas, Greendale, WI (US)

Correspondence Address: John M. Manion;RYAN KROMHOLZ & MANION, S.C., Post Office Box 26618, Milwaukee, WI, 53226-0618, US

(73) Assignee: Ancel Surgical R&D, Inc.

(21) Appl. No.: 11/493,988

(22) Filed: Jul. 27, 2006

Related U.S. Application Data
(60) Provisional application No. 60/704,506, filed on Aug. 1, 2005.

(51) Int. Cl. A61L 17/00

(52) U.S. Cl. 606/228

(57) ABSTRACT

A suture thread having a textured exposed surface, wherein the texture of the exposed surface enables the suture thread to easily move in a first longitudinal direction, but resist movement in a second longitudinal direction. The texture of the exposed surface being formed by imprinting or by cutting microscopic, angled incisions.

### Related Applications

This application claims the benefit of co-pending U.S. provisional patent application Ser. No. 60/704,506, filed 1 Aug. 2005.

### BACKGROUND OF THE INVENTION

The need for this invention arises from surgical practice, particularly surgical practice in which tissue must be held together for healing, as in a wound or incision. During healing of a wound or incision, tissue is typically held together with filament or suture thread.

In surgical practice, it is usually desired that suture thread be able to be pulled smoothly through tissue to be sutured. This characteristic reduces tissue trauma during the suturing procedure. Additionally, it is desired that suture thread maintains placement and resists reverse pull out, or slippage, once in place during healing. In the past, suture thread has been manufactured with purpose to enhance pull-through during suturing. For example, it is known to coat the exposed surface of suture thread with a lubricant to reduce surface friction during suturing. While this feature is helpful during suturing, it does not address slippage after suturing, in fact, this feature may actually increase slippage when it is not desired during healing. Thus, the abilities of suture thread to slide easily during suturing but also resist slippage after suturing are usually mutually exclusive. The very quality desired during suturing is that which is unwanted after suturing and during healing.

### SUMMARY OF THE INVENTION

It is therefore a principal object of the present invention to provide for a suture thread that slides easily in a first longitudinal direction during suturing, yet resists slippage in a second longitudinal direction after suturing and during healing.

To attain the object described, there is provided a suture thread having a longitudinal axis. The thread further includes an exposed surface that is configured to have a first coefficient of friction as the thread is moved in a first longitudinal direction, while having a second coefficient of friction as the thread is moved in a second longitudinal direction. For example, the first coefficient of friction may be adapted to ease the thread through the tissue to be sutured in a first longitudinal direction, while the second coefficient may be increased to therefore resist longitudinal movement in a second longitudinal direction.

By altering the texture of the suture thread exposed surface, the frictional properties of the thread may also be altered. For example, the exposed surface may be provided with a microscopic pattern that resembles fish scales. Alternatively, suture thread according to the present invention may include an exposed surface having incomplete cuts extending laterally from the exposed surface a predetermined distance toward the thread axis. The cuts may be preferably angled relative the longitudinal axis of the thread to form an overlapping, scale-like pattern comprised of overlapping members. The overlapping members remain flattened as the thread is moved in a first longitudinal direction, but create friction and resist movement as the thread is moved in an opposite longitudinal direction.

Patterns of the overlapping members as applied to the exposed surface of suture thread may be varied as to size and shape as desired by particular application. Further, if cuts are used to create the pattern, the cuts may also be varied as to location and depth to allow for variation thereof and flexibility in application. A method according to the present invention allows for the suture thread to be imprinted or cut to achieve the desired configuration. The method may also provide the claimed texture either in random or patterned configuration as well as covering the complete thread or a portion thereof, depending upon, among other things, the type of tissue being repaired. A further method includes the steps of suturing using the unique thread of the present invention.

### BRIEF DESCRIPTION OF THE DRAWINGS

FIG. 1 is a fragmentary side plan view of an embodiment of the suture thread according to the present invention and showing the thread moving in a first longitudinal direction.

FIG. 2 is a fragmentary side plan view of the suture thread shown in FIG. 1 but showing the thread moving in a second longitudinal direction with the trailing edges of the overlapping members providing added friction to thereby resist movement in the illustrated direction.

FIG. 3 is a fragmentary view of the thread illustrated in FIGS. 1 and 2 as it is moved in a first longitudinal direction, and in conjunction with a suture lock.

FIG. 4 is a view similar to that of FIG. 3, but illustrating the suture thread moving in a second longitudinal direction and resisting movement in the direction of the arrow.

FIG. 5 illustrates an alternative embodiment in which the suture thread may be further provided with an enlarged portion.

FIG. 6 illustrates a plurality of suture threads, similar to those shown in FIG. 5, but braided together.

FIG. 7 depicts a single suture thread and showing an enlarged portion.

FIG. 8 is a fragmentary side view of another alternative embodiment and illustrating suture thread wherein an enlarged portion is provided with overlapping members with the remainder being conventionally smooth.

### DESCRIPTION OF THE PREFERRED EMBODIMENT

Although the disclosure hereof is detailed and exact to enable those skilled in the art to practice the invention, the physical embodiments herein disclosed merely exemplify the invention, which may be embodied in other specific structures. While the preferred embodiment has been described, the details may be changed without departing from the invention, which is defined by the claims.

The present invention is directed to suture thread, and in particular suture thread having unidirectional pull capabilities. Specifically, the present invention pertains to suture thread having the capability to be pulled easily in one longitudinal direction, while resisting movement in an opposite longitudinal direction.

The purpose of the present invention is to provide a suture thread 10 with an exposed surface 12 having a first coefficient of friction when the thread 10 is moved in a first longitudinal direction and a second coefficient of friction when the thread 10 is moved in a second longitudinal direction. To accomplish this purpose, several different embodiments of suture thread are disclosed, which are directed to examples of suture thread having an exposed surface provided with overlapping, scale-like members 14. As will be described, the suture thread 10 with textured exposed surface 12 is adapted for use in a variety of settings.

As may be seen in the Figures, suture thread 10 preferably includes an exposed surface 12, having a predetermined texture, such as the scale-like members 14 shown. FIG. 1 illustrates a suture thread 10 with a texture according to the present invention, in which individual members 14 lay smoothly flat as the thread 10 is moved in the direction of arrow A. FIG. 2 illustrates the thread 10 moving in the longitudinal direction indicated by arrow B. As seen, thread movement in the direction of arrow B encourages the trailing edges 16 of respective members 14 to move laterally relative the exposed surface 12. As the trailing edges 16 move laterally relative the exposed surface 12, they serve to increase the coefficient of friction, thereby resisting thread 10 movement in the direction of arrow B.

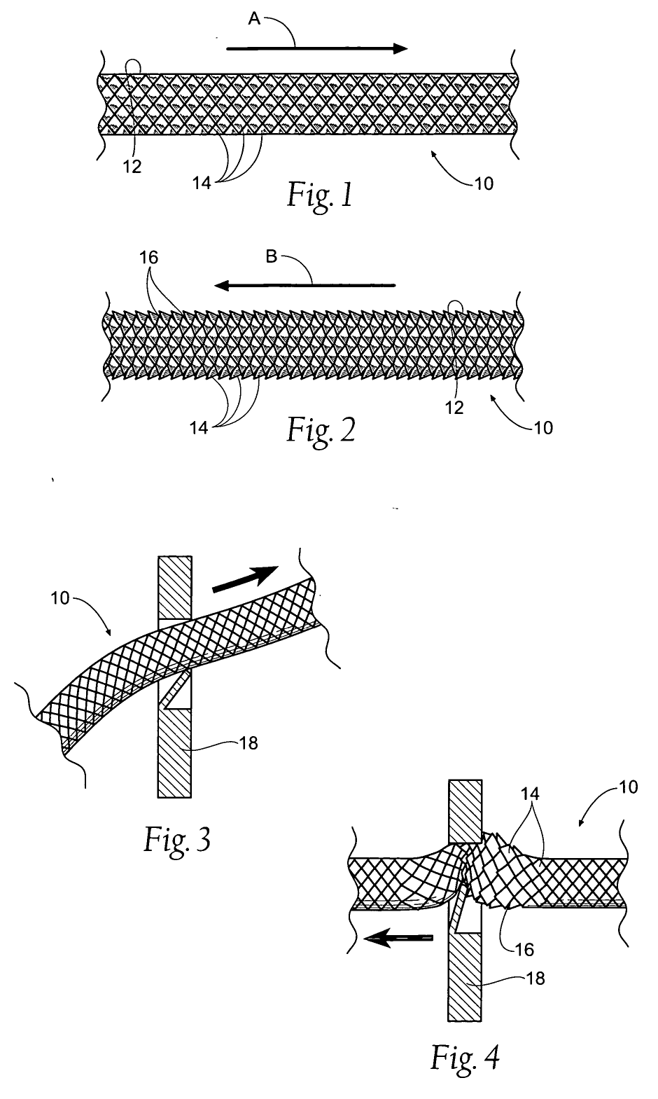

An application of the present suture thread 10 may be seen in FIGS. 3 and 4. As illustrated, the suture thread 10 may be used in conjunction with a suture lock 18. Although suture lock 18 is illustrated, it is to be understood that suture thread 10 may be used in conjunction with other known suture locks (not shown) to thereby assist in strengthening the suture.

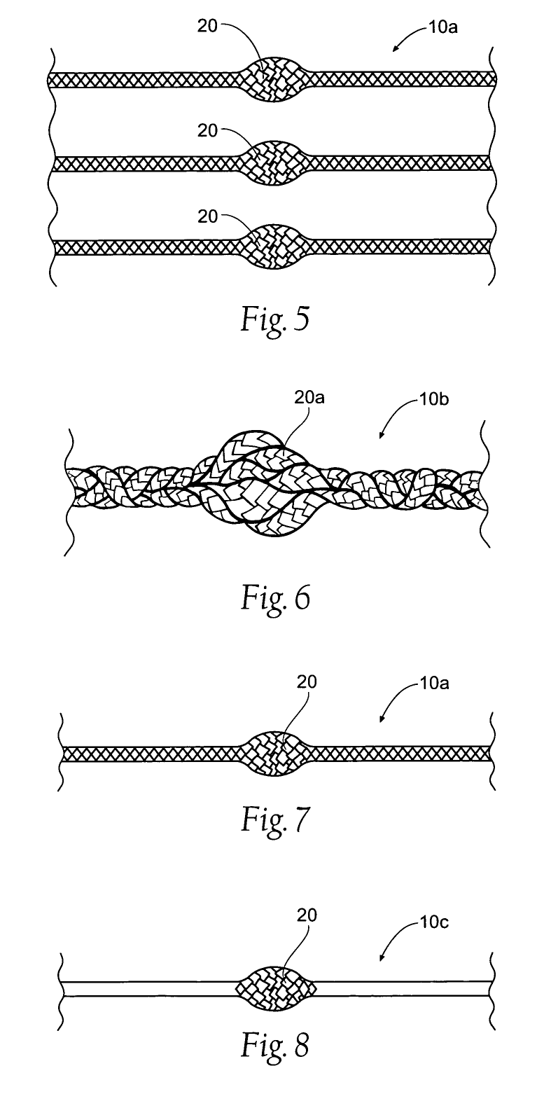

An alternative embodiment of the present suture thread 10 may be seen in FIGS. 5 and 7. As illustrated, at least one enlarged portion 20 may be provided. One, or a series, or plurality of spaced apart enlarged portions 20 may be formed in suture thread 10a. The needle (not shown) may, after passing through the tissue to be sutured, be drawn through an enlarged portion 20 a distance suitable to the surgeon making the stitch. The enlarged portion 20, in conjunction with the scale members 14, provides locking of the suture thread 10a in place without the necessity of tying a knot.

Another alternative embodiment suture thread 10b may be seen in FIG. 6. In this embodiment, the previously discussed suture thread 10a, having an enlarged portion 20, is additionally braided with multiple suture threads 10a, to thereby create a multi-strand combination suture thread 10b with enlarged portion 20a. The braided suture thread 10b offers added frictional resistance against pull out of the thread once passed through desired tissue to be sutured. Additionally the braided enlarged portion 20a offers added friction resistance against pull out after the needle (not shown) is drawn through portion 20a after making a stitch.

Referring now to FIG. 8, a design similar to that of FIGS. 5-7 is illustrated except that the enlarged portion 20 is covered in scale members 14, while the thread itself is conventionally smooth. This alternative embodiment is referenced generally as 10c, and offers added friction resistance against pull out after the needle (not shown) is drawn through portion 20 after making a stitch, while utilizing a conventionally smooth thread length.

It is to be understood that while the scale members 14 of the present invention may be imprinted or cut into the exposed surface 12, the size of each individual scale 14 may vary according to desired application according to an individual end user's requirements. Further, if the scales 14 are cut into the exposed surface 12, the depth of individual cuts may also vary to influence the size and shape of each trailing edge 16.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

1. A suture thread having an exposed surface, at least a portion of said exposed surface including a predetermined texture, said texture adapted to provide a first coefficient of friction as said thread is moved in a first longitudinal direction and a second coefficient of friction as said thread is moved in a second longitudinal direction.

2. The suture thread of claim 1 wherein said texture covers said exposed surface.

3. The suture thread of claim 1 further including at least one portion of enlarged thickness.

4. The suture thread of claim 3 wherein said texture covers said at least one portion of enlarged thickness.

5. The suture thread of claim 1 wherein said texture comprises a plurality of overlapping members, each member having a leading edge and a trailing edge.

6. The suture thread of claim 5 wherein said trailing edge is capable of lateral movement relative to said exposed surface.

7. The suture thread of claim 6 wherein said lateral movement is responsive to longitudinal movement of said suture thread.
